(19) United States
(12) Patent Application Publication
(10) Pub. No.: US 2026/0318860 A1
(43) Pub. Date: Oct. 15, 2026

(54) PROBE AND SYSTEM FOR MEASURING TRANS-EPITHELIAL ELECTRICAL RESISTANCE AND TRANS-EPITHELIAL POTENTIAL DIFFERENCE

(71) Applicant: EMD Millipore Corporation, Burlington, MA (US)

(72) Inventors: Joseph Lento, Hampton, NH (US); Spencer Keilich, Waltham, MA (US); Christian Carey, Medford, MA (US); Hai Pham, Fitchburg, MA (US); Paul Sydlowski, Danvers, MA (US)

(21) Appl. No.: 19/484,733

(22) PCT Filed: May 7, 2024
(86) PCT No.: PCT/US2024/028173

§ 371 Date: Nov. 14, 2025

Related U.S. Application Data
(60) Provisional application No. 63/503,028, filed on May 18, 2023.

(51) Int. Cl. G01N 27/327; G01K 7/24; G01N 33/487

(52) U.S. Cl. CPC G01N 27/327; G01K 7/24; G01N 33/48728

(57) ABSTRACT

A probe (30) for a trans-epithelial electrical resistance and trans-epithelial potential difference instrument (10) may include a temperature sensor configured to record the temperature in a well of a cell culture plate while collecting a TEER or TEPD measurement. The temperature sensor may be surface mounted on one of the legs (36) of the probe, may be disposed within a tube disposed proximate to one of the legs of the probe, or, when the probe legs are tubular, may be disposed within the conduit of one of the legs. The apical and basolateral legs may be formed as printed circuit board assemblies, where the sensing elements are subjected to an electroless nickel, immersion gold treatment process to coat the sensing elements in a layer or nickel and then a layer of gold. The apical and basolateral legs may also be formed from 304 stainless steel and may be tubular in shape.

### CROSS-REFERENCE TO RELATED APPLICATIONS

This present application claims priority to U.S. Provisional Patent Application Ser. No. 63/503,028, entitled “PROBE AND SYSTEM FOR MEASURING TRANS-EPITHELIAL ELECTRICAL RESISTANCE AND TRANS-EPITHELIAL POTENTIAL DIFFERENCE,” and filed on May 18, 2023, the disclosure of which is incorporated herein by reference in its entirety for all purposes.

### TECHNICAL FIELD

The present disclosure is directed generally toward an electrode probe and EVOM voltohmmeter that, in particular, is used for measuring Trans-Epithelial Electrical Resistance and Trans-Epithelial Potential Difference.

### BACKGROUND OF THE INVENTION

Trans-Epithelial Electrical Resistance (hereinafter “TEER”) is a commonly used non-destructive way to measure the confluency and barrier integrity of a cell monolayer. When measuring TEER, an AC electrical signal of known current (typically a 12.5 Hz square wave) is applied on either side of a cell monolayer. The voltage induced by this signal is transduced from the solution on either side of a cell monolayer. Because the voltage of this signal is measured and the current is known, ohms law (Voltage=Current*Resistance) can be used to calculate the electrical resistance of that monolayer. This technique is used both as a tool during the cell growth process to assess progress towards confluency of cell monolayers and to assess the end state of cells during a given experiment.

Trans-Epithelial Potential Difference (hereinafter “TEPD”) is a related technique which measures the potential difference (i.e., voltage) given off by a cell monolayer. The same equipment is used for TEPD and TEER measurements. Rather than applying a known current to the system, the voltage generated by the cells themselves is directly transduced from the solution on either side of a cell monolayer. This technique is less commonly used than TEER, and only applies to certain cell types which generate an electrical potential difference.

There are generally three categories of devices/systems for measuring TEER/TEPD. These systems include: (a) single-up instrument and probe systems; (b) single-up instrument and cup systems; and (c) automated high throughput systems. The present invention relates to single-up instrument and probe systems, and tangentially to automated high throughput systems.

Generally, single-up instrument and probe systems consist of two parts: (a) an instrument which reads measurements and may generate an electrical signal; and (b) a probe which interfaces with the cell culture setup and carries electrical signals to and from the instrument. These systems are capable of measuring one experiment (i.e., measure across the membrane of one well insert in a multi-well plate) at a time.

Conventional probes vary in overall shape, but consist of the same core features. Most conventional probes have two legs, an apical leg and a basolateral leg. When used, the apical leg sits in the inside of a well insert in a plate and the basolateral leg sits outside of the well insert but inside of the well itself. Moreover, each conventional probe typically has four sensor elements, where two elements may be used to detect voltage induced or generated by an experiment, and two elements may be used to apply a signal to the experiment for measurement of TEER. Furthermore, the conventional probe sensing element surfaces may be silver (either as a coating or base material). When used, customers/users are expected to soak their probes in sodium hypochlorite or hydrochloric acid to convert the surface to silver/silver chloride (Ag/AgCl). Customers/users must complete this process periodically in order to re-coat the surface when the AgCl is removed.

It is known that TEER measurements are temperature dependent. However, at present, conventional TEER measurement systems do not incorporate temperature measurement or controls into their platforms. While high end automated high throughput systems may take the temperature into account, this is generally accomplished by controlling the temperature of the environment in which the TEER measurements are conducted.

Automated high throughput systems are specialized platforms which often incorporate a chamber with environmental control and specialized custom consumables for use in their platform. Some automated high throughput systems may use existing well plates/inserts and perform measurements with multi-up probe heads. However, all high throughput systems on the market today are complex standalone systems. There are currently no existing add-ons to existing conventional single-up TEER platforms that are configured to transform the single-up TEER platform into a high throughput automated measurement system.

Therefore, it would be desirable incorporate a temperature measurement system (e.g., temperature sensor, thermistor, etc.) into at least one of the legs of probes to allow for TEER measurements in parallel with temperature measurements in order to normalize results of the measurements. It would further be desirable to formulate the sensing surface of the probe from a material other than silver/silver chloride in order to eliminate the need for periodic re-treatment of the probes with sodium hypochlorite or hydrochloric acid. In addition, it would be desirable to integrate networking capabilities into the TEER system to integrate the TEER system and TEER readings with a cloud-based analytical software. These and other objects are achieved by the embodiments disclosed herein.

### SUMMARY OF THE INVENTION

The present disclosure is directed toward a probe for a trans-epithelial electrical resistance and trans-epithelial potential difference instrument that may include a temperature sensor configured to record the temperature in a well of a cell culture plate while collecting a TEER or TEPD measurement. The temperature sensor may be surface mounted on one of the legs of the probe, may be disposed within a tube disposed proximate to one of the legs of the probe, or, when the probe legs are tubular, may be disposed within the conduit of one of the legs. The apical and basolateral legs may be formed as printed circuit board assemblies, where the sensing elements are subjected to an electroless nickel, immersion gold treatment process to coat the sensing elements in a layer or nickel and then a layer of gold. The apical and basolateral legs may also be formed from 304 stainless steel and may be tubular in shape.

According to one embodiment, a probe for a trans-epithelial electrical resistance and trans-epithelial potential difference instrument may include an upper housing, an electrode body coupled to the upper housing, an apical leg coupled to the electrode body, a basolateral leg coupled to the electrode body, and a temperature sensor disposed proximate to the apical leg or the basolateral leg.

In some instances, the temperature sensor may be surface mounted on the apical leg, and the temperature sensor may be coated with an electrically isolating coating. In some other instances, the temperature sensor may be an encapsulated thermistor or a thermocouple. In some further instances, the apical leg may have an upper end, an opposite lower end, and at least one sensing element disposed proximate to the lower end. In some even further instances, the temperature sensor may be disposed proximate to the at least one sensing element.

Still further, in some instances, the probe may further include a tube disposed in proximity to the apical leg, where the tube may have an upper end and a lower end, and where the lower end of the tube may be disposed in proximity to the lower end of the apical leg. Furthermore, the temperature sensor may be disposed within the lower end of the tube. Even further, the tube may be a stainless steel tube. In some further instances, the tube may be coupled to the electrode body, where, in some other instances, the tube may be coupled to the apical leg. In addition, the apical leg may contain a channel and the tube may be disposed within the channel of the apical leg.

In some even further instances, the instrument may comprise a network interface unit configured to send measurements taken via the probe to an analytical server over a network.

In another embodiment, a probe for a trans-epithelial electrical resistance and trans-epithelial potential difference instrument may include an upper housing, a electrode body coupled to the upper housing, an apical leg coupled to the electrode body, and a basolateral leg coupled to the electrode body, where the apical leg and the basolateral leg are printed circuit board assemblies.

In some instances, the apical leg may have an upper end, an opposite lower end, and at least one copper sensing element disposed proximate to the lower end. In some further instances, the basolateral leg may also have an upper end, an opposite lower end, and at least one copper sensing element disposed proximate to the lower end. In some even further instances, at least one copper sensing elements of the apical leg and the basolateral leg may be coated with a layer of nickel and a layer of gold via an electroless nickel, immersion gold treatment process. In still some further instances, the probe may include a temperature sensor disposed proximate to the apical leg or the basolateral leg.

In even some further instances, the instrument may comprise a network interface unit configured to send measurements taken via the probe to an analytical server over a network.

In yet another embodiment, a probe for a trans-epithelial electrical resistance and trans-epithelial potential difference instrument may include an upper housing, a electrode body coupled to the upper housing, a tubular apical leg coupled to the electrode body, and a tubular basolateral leg coupled to the electrode body, wherein the tubular apical leg and the tubular basolateral leg constructed from an electrochemically inert and stable material.

In some instances, the tubular apical leg and the tubular basolateral leg may be constructed from stainless steel. In some even further instances, the tubular apical leg and the tubular basolateral leg may be constructed from citric passivated 304 stainless steel. In addition, the tubular apical leg may have an upper end, an opposite lower end, and a conduit spanning from the upper end to the lower end. Furthermore, in some additional instances, a temperature sensor may be disposed within the conduit of the tubular apical leg proximate to the lower end.

In even some further instances, the instrument may comprise a network interface unit configured to send measurements taken via the probe to an analytical server over a network.

Other systems, devices, apparatuses, methods, features, and advantages will be, or will become, apparent to one with skill in the art upon examination of the following figures and detailed description. All such additional systems, devices, arrangements, mechanisms, assemblies, apparatuses, methods, features, and advantages are included within this description, are within the scope of the claimed subject matter.

### BRIEF DESCRIPTION OF THE DRAWINGS

The apparatuses, systems, assemblies, devices, and components presented herein may be better understood with reference to the following drawings and description. It should be understood that some elements in the figures may not necessarily be to scale and that emphasis has been placed upon illustrating the principles disclosed herein. In the figures, like-referenced numerals designate corresponding parts/steps throughout the different views.

FIG. 1 illustrates a perspective view of an example embodiment of a single-up TEER instrument, including a probe, in accordance with the present invention.

FIG. 2A illustrates a 24 well cell culture plate configured to support cell growth and be utilized with a single-up TEER instrument like that illustrated in FIG. 1.

FIG. 2B illustrates a 96 well cell culture plate configured to support cell growth and be utilized with a single-up TEER instrument like that illustrated in FIG. 1.

FIG. 3 illustrates a schematic illustration of a probe of a single-use TEER instrument inserted into a well of one of the cell culture plates illustrated in FIG. 1, 2A, or 2B in order to measure the transepithelial electrical resistance.

FIGS. 4A-4C illustrate perspective views of a first embodiment of a probe of a single-use TEER instrument in accordance with the present invention, where a temperature sensor is integrated into one of the legs of the probe, and where the legs of the probe are printed circuit board assemblies.

FIGS. 5A-5D illustrate perspective views of a second embodiment of a probe of a single-use TEER instrument in accordance with the present invention, where a temperature sensor is disposed within a tube disposed in proximate to one of the legs of the probe, and where the legs of the probe are printed circuit board assemblies.

FIG. 5E illustrates a cross-sectional view of the probe illustrated in FIGS. 5A-5D, where the cross-sectional is taken along line 5E-5E in FIG. 5B.

FIGS. 6A-6D illustrate perspective views of a third embodiment of a probe of a single-use TEER instrument in accordance with the present invention, where the legs of the probe are tubular, and where a temperature sensor is disposed within the tube of one of the legs of the probe.

FIG. 6E illustrates a cross-sectional view of the probe illustrated in FIGS. 6C and 6D, where the cross-sectional is taken along line B-B in FIG. 6C.

FIG. 7 illustrates a flowchart of the method for constructing the legs of the probes illustrated in FIGS. 4A-4C and FIGS. 5A-5E, and in accordance with the present invention.

FIG. 8 illustrates a block diagram of a network environment in which embodiments of a single-up TEER instrument communicate with a cloud analytical tool, according to an example embodiment.

FIG. 9 illustrates is a block diagram of a controller for a single-up TEER instrument configured to perform the techniques described herein, according to an embodiment.

FIGS. 10A-10C illustrate example embodiments of graphical user interfaces for the cloud based analytical tool that utilizes the data acquired from a single-up TEER instrument in accordance with the present invention.

### DETAILED DESCRIPTION OF THE INVENTION

In the following detailed description, reference is made to the accompanying figures which form a part hereof wherein like numerals designate like parts throughout, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the description herein. Alternate embodiments of the present disclosure and their equivalents may be devised without parting from the spirit or scope of the present disclosure. It should be noted that any discussion herein regarding “one embodiment,” “an embodiment,” “an exemplary embodiment,” and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

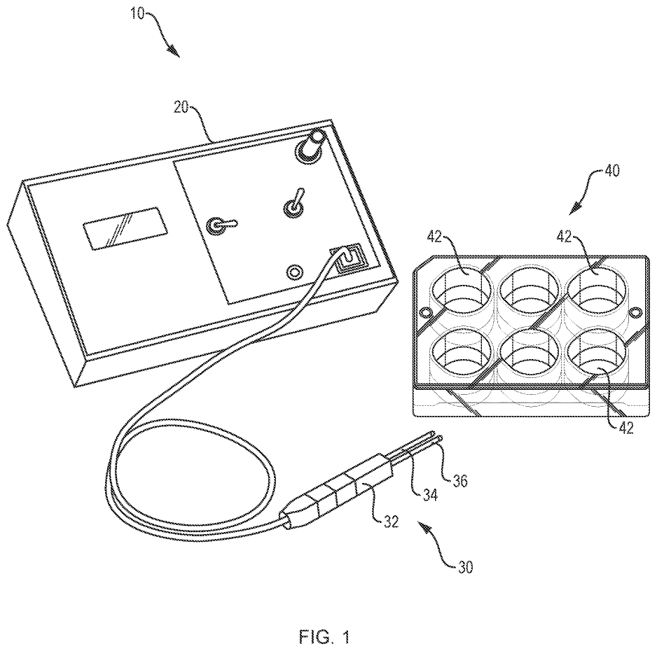

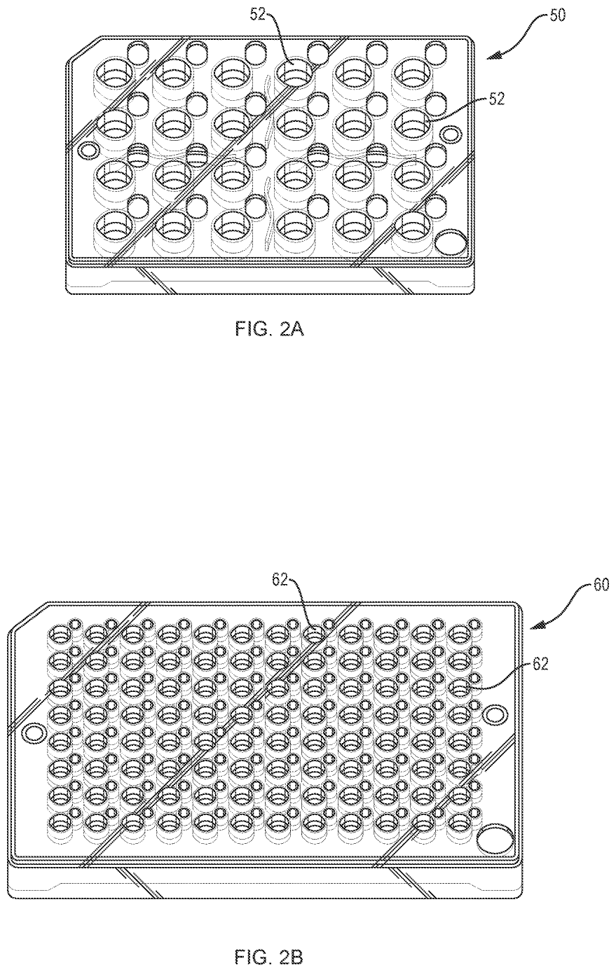

Turing to FIG. 1, illustrated is an example embodiment of a voltohmmeter or single-up TEER system (hereinafter “TEER system”) 10 configured to measure the trans-epithelial electrical resistance (hereinafter “TEER”) of a cell culture well that may contain cells. The TEER system may contain a measurement meter 20 and a probe 30 that is connected with the measurement meter 20, where the probe 30 may be configured for insertion of into a cell culture well 42 of a cell culture plate 40, which is also depicted in FIG. 1. As explained in further detail below, the probe 30 may contain an electrode body 32, an apical leg 34, and a basolateral leg 36. While the cell culture plates 40 shown in FIG. 1 contains 6 wells, FIGS. 2A and 2B illustrate other embodiments of cell culture plates 50, 60. The cell culture plate 50 illustrated in FIG. 2A is a 24-well plate (i.e., a cell culture plate having 24 wells 52), while the cell culture plate 60 illustrated in FIG. 2B is a 96-well plate (i.e., a cell culture plate having 96 wells 62).

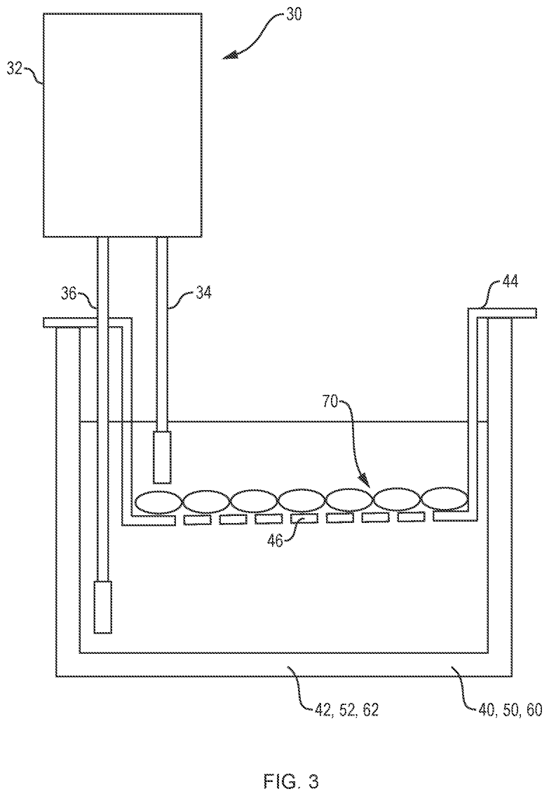

FIG. 3 illustrates a schematic embodiment of a probe 20 measuring the TEER of the cells 70 in a well 42, 52, 62 of a cell culture plate 40, 50, 60. As further illustrated, a hanging insert 44 is placed within the well 42, 52, 62 of a cell culture plate 40, 50, 60, where the bottom surface 46 of the hanging insert 44 contains a semi-permeable membrane upon which the cells 70 grow. When the probe 20 is inserted into the well 42, 52, 62, the apical leg 34 is disposed within the hanging insert 44 (i.e., within the upper compartment of the well 42, 52, 62), while the basolateral leg 36 is disposed within the well 42, 52, 62, but outside of the hanging insert 44 (i.e., within the lower compartment of the well 42,52, 62). An AC voltage may be applied between the legs 34, 36 of the probe 30, where the electrical resistance of the cell barrier tissue is measured/calculated by the measurement meter 10. The electrical resistance, or TEER, is a quantitative parameter of the integrity of a cell monolayer through its ionic conductance. Thus, the TEER value rises as the cells proliferate and lowers when the barrier is compromised.

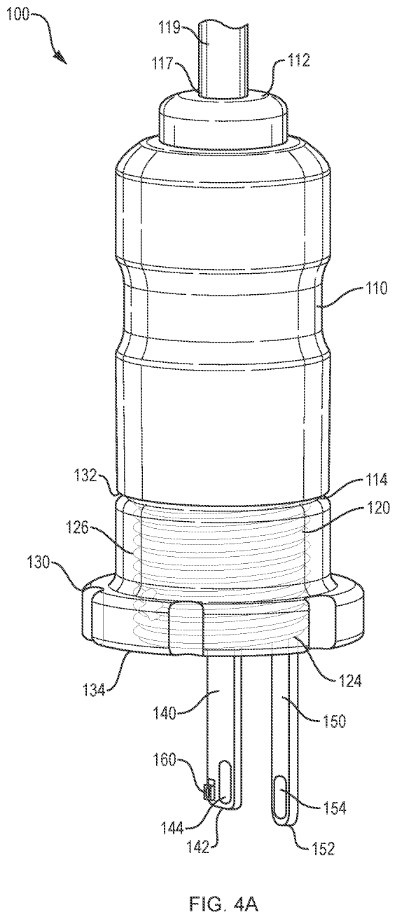

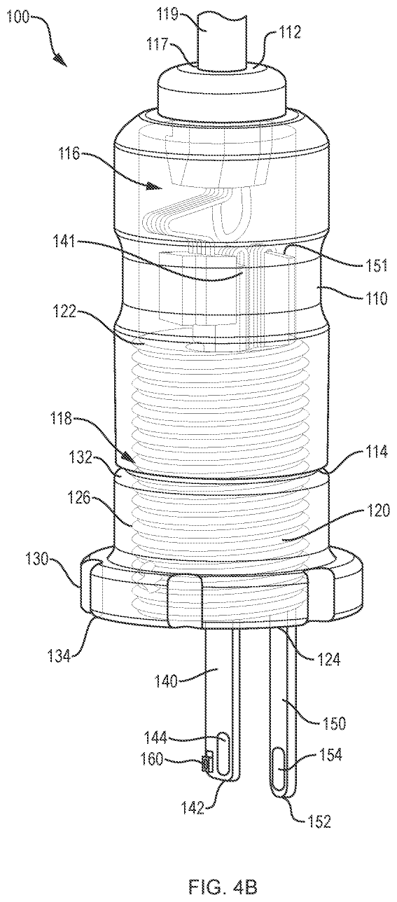

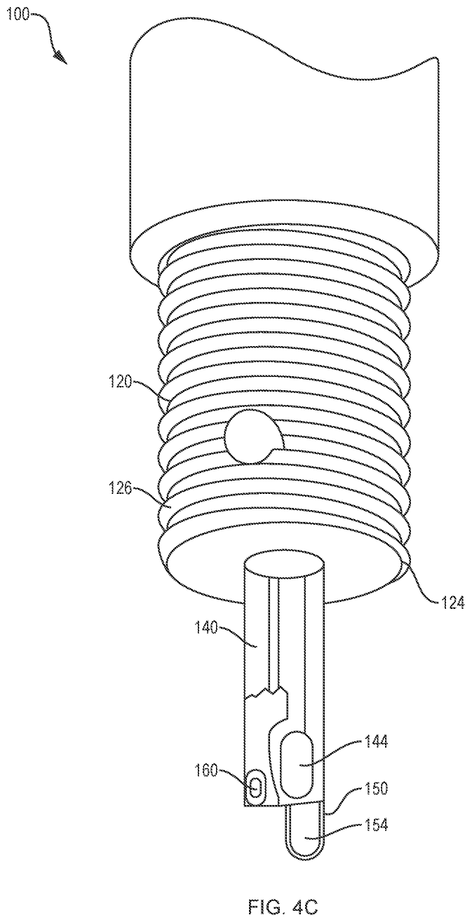

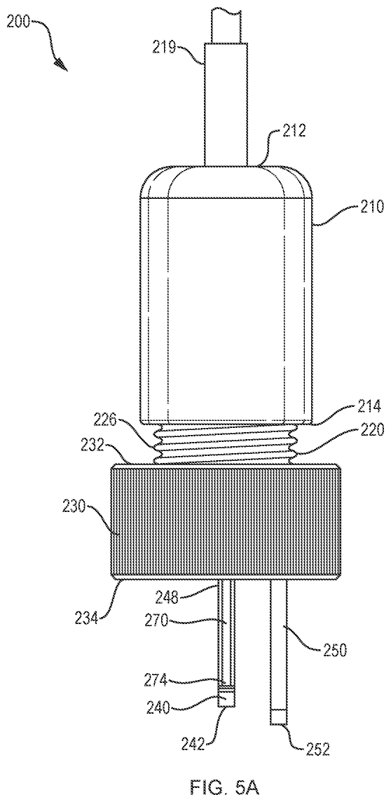

Turning to FIGS. 4A-4C, illustrated are various perspective views of a first embodiment of a probe 100 of a TEER system 10 in accordance with the present invention. The probe 100 may be utilized for measuring the TEER of a well 52 of a 24-well cell culture plate 50. In other words, the probe 100 may be a 24-well probe. More specifically, the probe 100 includes a top housing 110 having a top end 112 and an opposite bottom end 114, where the top housing 110 defines an interior cavity 116 accessible via a top opening 117 in the top end 112 and a bottom opening 118 in the bottom end 114 of the top housing 110. The top opening 117 may be smaller in size than the bottom opening 118, and a cable 119 may be disposed within the top opening 117 such that the cable 119 extends from the top end 112 of the top housing 110.

Continuing with FIGS. 4A-4C, the probe 100 further includes an electrode body 120 having a top end 122, an opposite bottom end 124, and a threaded sidewall 126 spanning from the top end 122 to the bottom end 124. As further illustrated, the bottom end 114 of the top housing 110 may be threaded onto the top end 122 of the electrode body 120 such that the bottom end 114 of the top housing 110 at least partially encircles the top end 122 of the electrode body 120 (and such that the electrode body 120 seals the interior cavity 116 of the top housing 110 at the bottom opening 118). Threaded onto the bottom end 124 of the electrode body 120 may be an electrode depth controller 130. The top end 132 of the electrode depth controller 130 may face or be in abutment with the bottom end 114 of the top housing 110 of the probe 100. The electrode depth controller 130 may be threaded onto the electrode body 120 to any suitable position for operation of the probe 100, including, but not limited to, the bottom end 134 of the electrode depth controller 130 being aligned with the bottom end 124 of the electrode body 120, the bottom end 134 of the electrode depth controller 130 being disposed higher than the bottom end 124 of the electrode body 120, or the bottom end 134 of the electrode depth controller 130 being disposed lower than the bottom end 124 of the electrode body 120. In the embodiment illustrated in FIG. 4A, the electrode depth controller 130 may be transparent, while in other embodiments the electrode depth controller 130 may be translucent or opaque.

Extending through the electrode body 120 may be an apical leg 140 and a basolateral leg 150. As illustrated, and because it is designed to be disposed within the upper compartment of a well 52 of the cell culture plate 50, the apical leg 140 may be shorter in length than the basolateral leg 150. Both legs 140, 150 may include an upper end 141, 151 (best shown in FIG. 4B) located within the interior cavity 116 of the top housing 110, and a lower end 142, 152, respectively. The apical leg 140 may extend from a center of the bottom end 124 of the electrode body 120 such that the apical leg 140 and the electrode body 120 are coaxial with one another. The basolateral leg 150 may extend from the bottom end of the electrode body 120 proximate to the threaded sidewall 126 of the electrode body 120.

As further illustrated, the apical leg 140 and the basolateral leg 150 may both be formed as printed circuit board assemblies (hereinafter “PCBA's”), where the copper or electrical sensing elements 144, 154, respectively, are disposed on the legs 140, 150 proximate to the lower ends 142, 152, respectively. In addition, the electrical sensing elements 144, 154 may be disposed on the outer and inner sides of the legs 140, 150. According to one embodiment, the electrical sensing elements 144, 154 may be formed as exposed copper pads that are coated with nickel and then gold using an electroless nickel, immersion gold (hereinafter “ENIG”) PCB pad treatment process. In another embodiment, the electrical sensing elements 144, 154 may be formed as exposed copper pads that are coated with nickel and then gold using an electroplated nickel/gold (hereinafter “hard gold”) PCB pad treatment process that results in a hard gold plating of the electrical sensing elements 144, 154. Both pad treatment processes result in at least the surface of the electrical sensing elements 144, 154 being a gold surface. Furthermore, treating the electrical sensing elements 144, 154 with either pad treatment process provides a flat, uniform surface for the electrical sensing elements 144, 154, which results in the electrical sensing elements 144, 154 being highly resistant to corrosion and oxidation.

As further illustrated in FIGS. 4A-4C, the probe 100 may include a temperature sensor or thermistor 160 integrated (e.g., surface mounted) onto one of the legs 140, 150 proximate to the lower end 142, 152 of the legs 140, 150, respectively. In the illustrated embodiment, the temperature sensor 160 is disposed on the apical leg 140 of the probe 100, and proximate to the electrical sensing element 144 disposed on the outer side of the apical leg 140. Moreover, the temperature sensor 160 may be conformally coated to electrically isolate the temperature sensor 160 from an experiment under test. While a surface mounted thermistor 160 is shown in FIGS. 4A-4C, the temperature sensor may be any other type of temperature sensor including, but not limited to, an encapsulated thermistor, a thermocouple, or any other temperature sensing element.

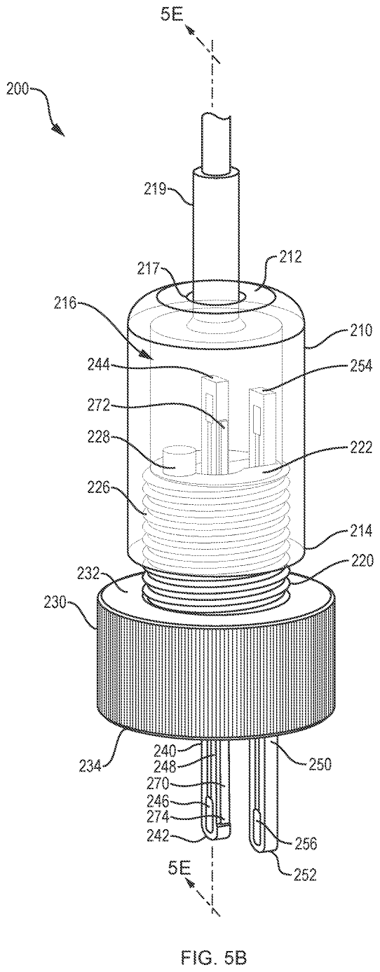

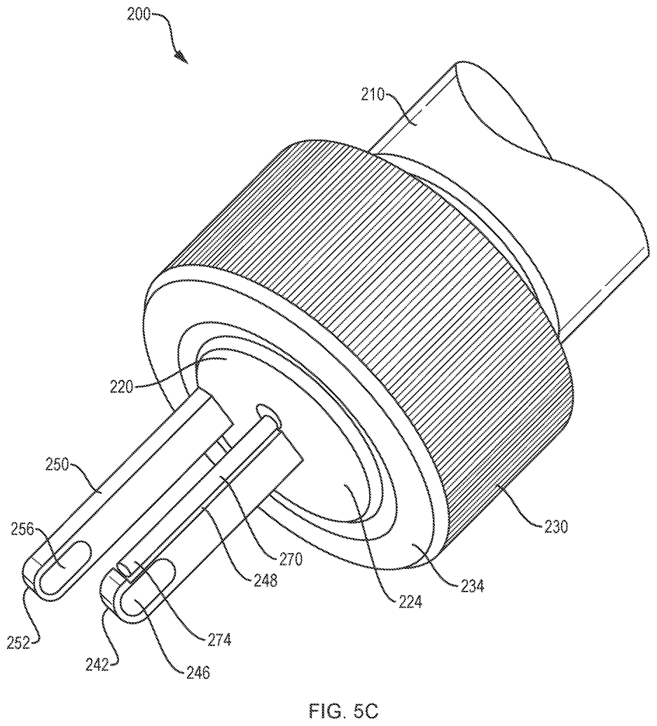

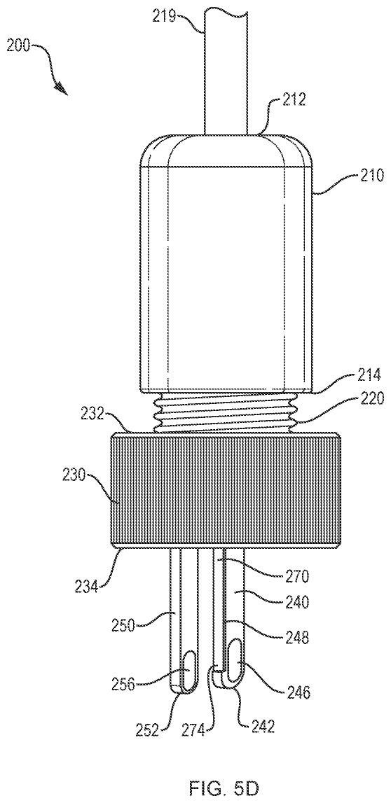

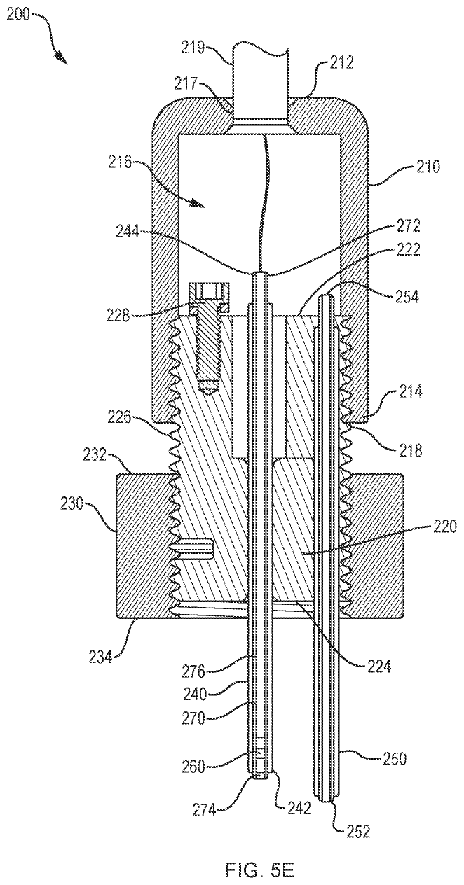

Turning to FIGS. 5A-5E, illustrated are various perspective views and one cross-sectional view (FIG. 5E) of a second embodiment of a probe 200 of a TEER system 10 in accordance with the present invention. Like the first embodiment of the probe 100, the second embodiment of the probe 200 may be a 24-well probe that is designed for measuring the TEER of a well 52 of a 24-well cell culture plate 50. Similar to the first embodiment of the probe 100, the probe 200 includes a top housing 210 having a top end 212 and an opposite bottom end 214, where the top housing 210 defines an interior cavity 216 accessible via a top opening 217 in the top end 212 and a bottom opening 218 in the bottom end 214 of the top housing 210. The top opening 217 may be smaller in size than the bottom opening 218, and a cable 219 may be disposed within the top opening 217 such that the cable 219 extends from the top end 212 of the top housing 210.

Continuing with FIGS. 5A-5E, the probe 200 also includes an electrode body 220 having a top end 222, an opposite bottom end 224, and a threaded sidewall 226 spanning from the top end 222 to the bottom end 224. As further illustrated, the bottom end 214 of the top housing 210 may be threaded onto the top end 222 of the electrode body 220 such that the bottom end 214 of the top housing 210 at least partially encircles the top end 222 of the electrode body 220 (and such that the electrode body 220 seals the interior cavity 216 of the top housing 210 at the bottom opening 218). As best illustrated in FIG. 5E, a grounding element 228 may be coupled to (i.e., a screw threaded/screwed onto, etc.) to top end 222 of the electrode body 220.

As further illustrated in FIGS. 5A-5E, and similar to the first embodiment of the probe 100, the second embodiment of the probe 100 may include an electrode depth controller 230 that may be threaded onto the bottom end 224 of the electrode body 220. The top end 232 of the electrode depth controller 230 may face or be in abutment with the bottom end 214 of the top housing 210 of the probe 200. The electrode depth controller 230 may be threaded onto the electrode body 220 to any suitable position for operation of the probe 200, including, but not limited to, the bottom end 234 of the electrode depth controller 230 being aligned with the bottom end 224 of the electrode body 220, the bottom end 234 of the electrode depth controller 230 being disposed higher than the bottom end 224 of the electrode body 220, or the bottom end 234 of the electrode depth controller 230 being disposed lower than the bottom end 224 of the electrode body 220. The electrode depth controller 230 may be transparent, translucent, or opaque, and the outer sidewall surface may be knurled.

Extending through the electrode body 220 may be an apical leg 240 and a basolateral leg 250. As best illustrated in FIGS. 5C and 5E, and because it is designed to be disposed within the upper compartment of a well 52 of the cell culture plate 50, the apical leg 240 may be shorter in length than the basolateral leg 250. Both legs 240, 250 may include a lower end 242, 252, and an upper end 244, 254, respectively, where the upper ends 244, 254 may be disposed within the interior cavity 216 of the top housing 210 (as best shown in FIG. 5E). The apical leg 240 may extend from a center of the bottom end 224 of the electrode body 220 such that the apical leg 240 and the electrode body 220 are coaxial with one another. The basolateral leg 250 may extend from the bottom end 224 of the electrode body 220 proximate to the threaded sidewall 226 of the electrode body 220.

As further illustrated, and similar to the first embodiment of the probe 100, the apical and basolateral legs 240, 250 of the second embodiment of the probe 200 may both be formed as PCBA's, where the copper or electrical sensing elements 246, 256, respectively, are disposed on the legs 240, 250 proximate to the lower ends 242, 252, respectively. In addition, the electrical sensing elements 246, 256 may be disposed on the outer and inner sides of the legs 240, 250. Like the electrical sensing elements 144, 154 of the first embodiment of the probe 100, the electrical sensing elements 246, 256 may be formed as exposed copper pads that are coated with nickel and then gold using either the ENIG PCB pad treatment process or the hard gold PCB pad treatment process, which leave the surface of the legs 240, 250 with a gold surface at least where the electrical sensing elements 246, 256 are located. Furthermore, treating the electrical sensing elements 246, 256 with either pad treatment process provides a flat, uniform surface for the electrical sensing elements 144, 154, which results in the electrical sensing elements 246, 256 being highly resistant to corrosion and oxidation.

Where the second embodiment of the probe 200 mainly differs from the first embodiment of the probe 100 is the integration of the temperature sensor/thermistor 260. In the embodiment illustrated in FIGS. 5A-5E, the temperature sensor 260 may be disposed within a tube 270 that may be coupled to the apical leg 240, rather than the temperature sensor 260 being disposed on a surface of the apical leg 240. The temperature sensor 260 may be any type of temperature sensor, including, but not limited to, a surface mounted thermistor, an encapsulated thermistor, a thermocouple, or any other temperature sensing element. More specifically, the tube 270 may include a top end 272, an opposite lower end 274, and a channel or conduit 276 running through the tube 270 from the top end 272 to the lower end 274. In some embodiments, the tube 270 may be a stainless steel tube. Furthermore, the temperature sensor 260 may be encapsulated within the conduit 276 of the tube proximate to the lower end 274 of the tube 270. The tube 270 may be coupled to the apical leg 240 and/or the electrode body 220 via bonding, adhesives, glues, epoxy, or any known coupling mechanism. In the embodiment shown in FIGS. 5A-5E, the apical legs 240 may contain a depression/cutout/channel 248, where the tube 270 may be coupled to the apical leg 240 such that the tube 270 is at least partially disposed within the channel 248, and such that the lower end 274 of the tube 270 and the temperature sensor 260 are disposed in proximity to the electrical sensing elements 246. In other embodiments, the apical leg 240 may not contain any channels 248, and the tube 270 with the temperature sensor 260 may be coupled to or disposed along any side or edge of the apical leg 240. In some embodiments, the tube 270 may be a stainless-steel tube, while, in other embodiments, the tube 270 may be constructed from any other suitable material that electrically insulates the temperature sensor 260 but enables the temperature sensor 260 to detect the temperature within the well 52 of the cell culture plate 50.

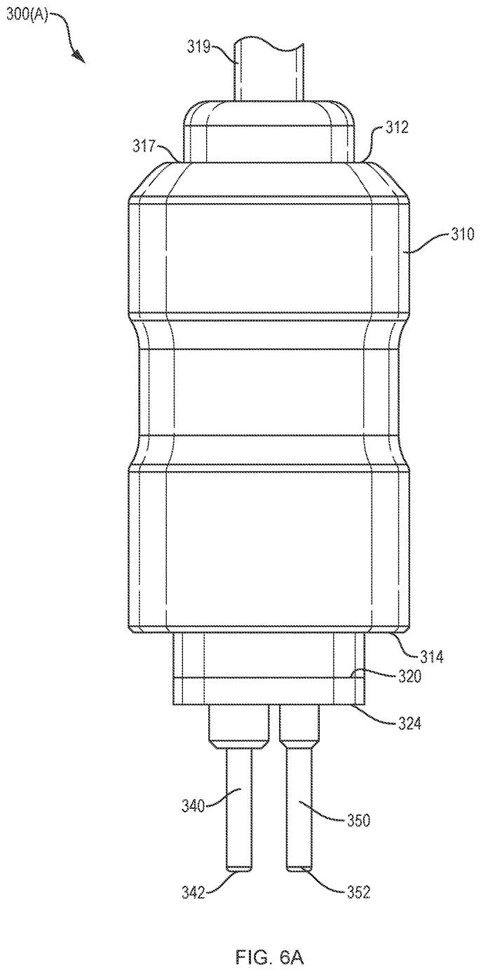

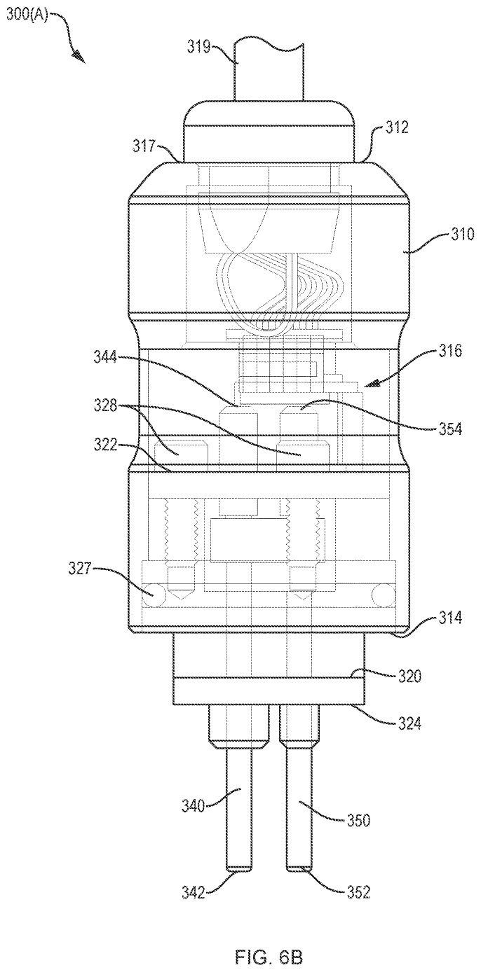

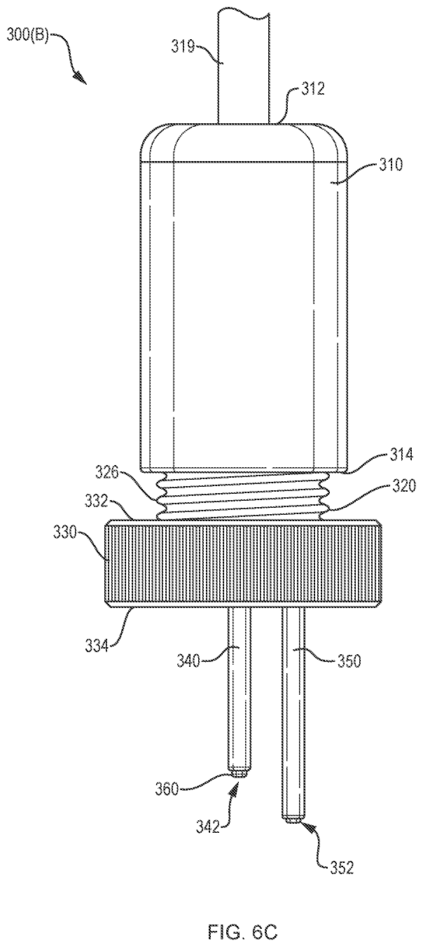

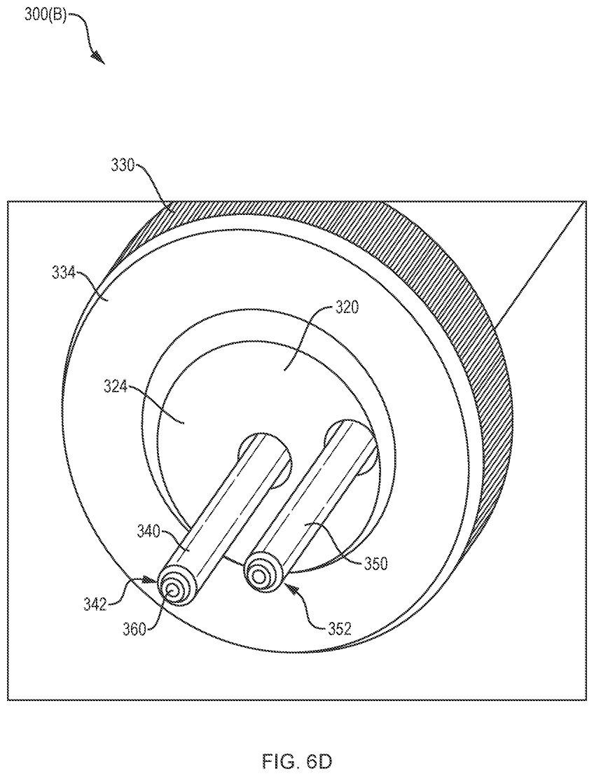

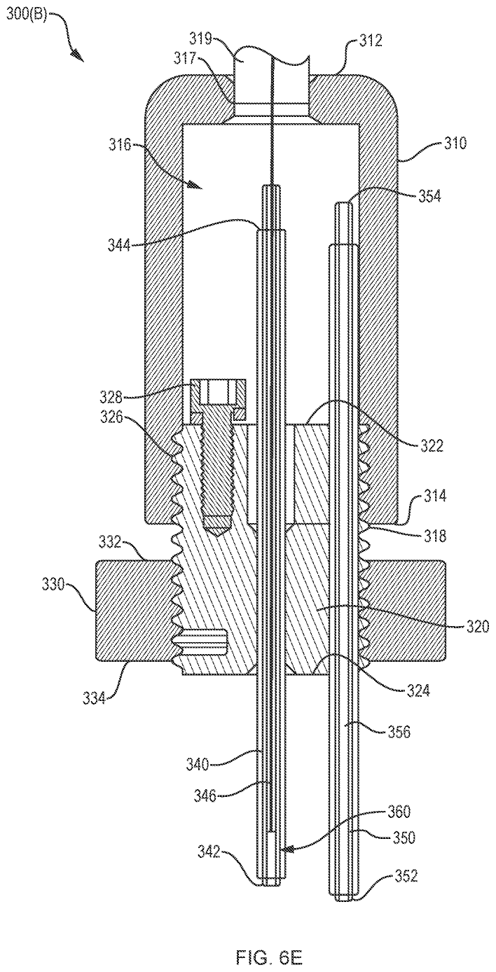

With reference to FIGS. 6A-6E, illustrated are various perspective views and one cross-sectional view (FIG. 6E) of a third embodiment and a fourth embodiment of a probe 300(A) and 300(B) of a TEER system 10 in accordance with the present invention. Unlike the first and second embodiments of the probe 100, 200, the third and fourth embodiments of the probe 300(A), 300(B) may be 96-well probes that are designed for measuring the TEER of a well 62 of a 96-well cell culture plate 60.

As best illustrated in FIGS. 6A and 6B, the third embodiment of the probe 300(A) includes a top housing 310 having a top end 312 and an opposite bottom end 314, where the top housing 310 defines an interior cavity 316 accessible via a top opening 317 in the top end 312 and a bottom opening 318 in the bottom end 314 of the top housing 310. The top opening 317 may be smaller in size than the bottom opening 318, and a cable 319 may be disposed within the top opening 317 such that the cable 319 extends from the top end 312 of the top housing 310.

Continuing with FIGS. 6A and 6B, the probe 300(A) also includes an electrode body 320 having a top end 322 and an opposite bottom end 324. However, unlike the previous two embodiments of the probe 100, 200, the electrode body 320 of the third embodiment of the probe 300(A) contains a smooth sidewall 326 spanning from the top end 322 to the bottom end 324 rather than a threaded sidewall. As best illustrated in FIG. 6B, the bottom end 314 of the top housing 310 may slide over the top end 322 of the electrode body 320 such that the bottom end 314 of the top housing 310 at least partially encircles the top end 322 of the electrode body 320 (and such that the electrode body 320 seals the interior cavity 316 of the top housing 310 at the bottom opening 318). As best illustrated in FIG. 6B, disposed within the bottom opening 318 of the top housing 310 of the probe 300(A) may be a gasket 327 that serves to form a friction fit of the top housing 310 with the electrode body 320. As also illustrated in FIG. 6B, and like the second embodiment of the probe 200, one or more grounding elements 328 may be coupled to (i.e., a screw threaded/screwed onto, etc.) to top end 322 of the electrode body 320.

The fourth embodiment of the probe 300(B) may be more similar in structure to the first two embodiments of the probe 100, 200 than the third embodiment of the probe 300(A). The fourth embodiment of the probe 300(B) may include a top housing 310 having a top end 312 and an opposite bottom end 314, where the top housing 310 defines an interior cavity 316 accessible via a top opening 317 in the top end 312 and a bottom opening 318 in the bottom end 314 of the top housing 310. The top opening 317 may be smaller in size than the bottom opening 318, and a cable 319 may be disposed within the top opening 317 such that the cable 319 extends from the top end 312 of the top housing 310.

Continuing with FIGS. 6C-6E, the probe 300(B) also includes an electrode body 320 having a top end 322, an opposite bottom end 324, and a threaded sidewall 326 spanning from the top end 322 to the bottom end 324. As further illustrated, the bottom end 314 of the top housing 310 may be threaded onto the top end 322 of the electrode body 320 such that the bottom end 314 of the top housing 310 at least partially encircles the top end 322 of the electrode body 320 (and such that the electrode body 320 seals the interior cavity 316 of the top housing 310 at the bottom opening 318). As best illustrated in FIG. 6E, and like the second embodiment of the probe 200, a grounding element 328 may be coupled to (i.e., a screw threaded/screwed onto, etc.) to top end 322 of the electrode body 320.

As further illustrated in FIGS. 6C-6E, unlike the third embodiment of the probe 300(A), and similar to the first and second embodiments of the probe 100, 200, the fourth embodiment of the probe 300(B) may include an electrode depth controller 330 that may be threaded onto the bottom end 324 of the electrode body 320. The top end 332 of the electrode depth controller 330 may face or be in abutment with the bottom end 314 of the top housing 310 of the probe 300(B). In addition, the electrode depth controller 330 may be threaded onto the electrode body 320 to any suitable position for operation of the probe 300(B), including, but not limited to, the bottom end 334 of the electrode depth controller 330 being aligned with the bottom end 324 of the electrode body 320, the bottom end 334 of the electrode depth controller 330 being disposed higher than the bottom end 324 of the electrode body 320, or the bottom end 334 of the electrode depth controller 330 being disposed lower than the bottom end 324 of the electrode body 320. The electrode depth controller 330 may be transparent, translucent, or opaque, and the outer sidewall surface may be knurled.

FIGS. 6A-6E further illustrate that both the third and fourth embodiments of the probe 300(A), 300(B) have an apical leg 340 and a basolateral leg 350 that extend through the electrode body 320. Because it is designed to be disposed within the upper compartment of a well 62 of the cell culture plate 60, the apical leg 340 may be shorter in length than the basolateral leg 350. As best illustrated in FIG. 6C, both legs 340, 350 may include a lower end 342, 352, and an upper end 344, 354, respectively, where the upper ends 344, 354 may be disposed within the interior cavity 316 of the top housing 310. Like the other embodiments of the probes 100, 200, the apical leg 340 of the third and fourth embodiments of the probe 300(A), 300(B) may extend from a center of the bottom end 324 of the electrode body 320 such that the apical leg 340 and the electrode body 320 are coaxial with one another. The basolateral leg 350 may extend from the bottom end 324 of the electrode body 320 proximate to the threaded sidewall 326 of the electrode body 320. However, unlike the first and second embodiments of the probe 100, 200, the apical and basolateral legs 340, 350 of the third and fourth embodiments of the probe 300(A), 300(B) may be constructed from tubing that is, preferably, but not limited to, constructed from electrochemically inert and stable materials. In one example embodiment, the apical and basolateral legs 340, 350 are constructed from 304 stainless steel tubing. More preferably, the 304 stainless steel tubing of the legs 340, 350 may be citric passivated, which enables the stainless steel to have an electrochemically inert nature similar to that of gold. As best illustrated in FIG. 6E, where the representation of the apical and basolateral legs 340, 350 may equally represent the third embodiment of the probe 300(A) and the fourth embodiment of the probe 300(B), the apical leg 340 may include a conduit 346 extending from the upper end 344 to the lower end 342, while the basolateral leg 350 may also include a conduit 356 extending from the upper end 354 to the lower end 352.

The third and fourth embodiments of the probe 300(A), 300(B) also include an integrated temperature sensor/thermistor 360. As best illustrated in FIG. 6E, the temperature sensor 360 may be disposed within the conduit 346 of the tubular apical leg 340 proximate to the lower end 342 of the apical leg 340. The temperature sensor 360 may be any type of temperature sensor, including, but not limited to, a surface mounted thermistor, an encapsulated thermistor, a thermocouple, or any other temperature sensing element.

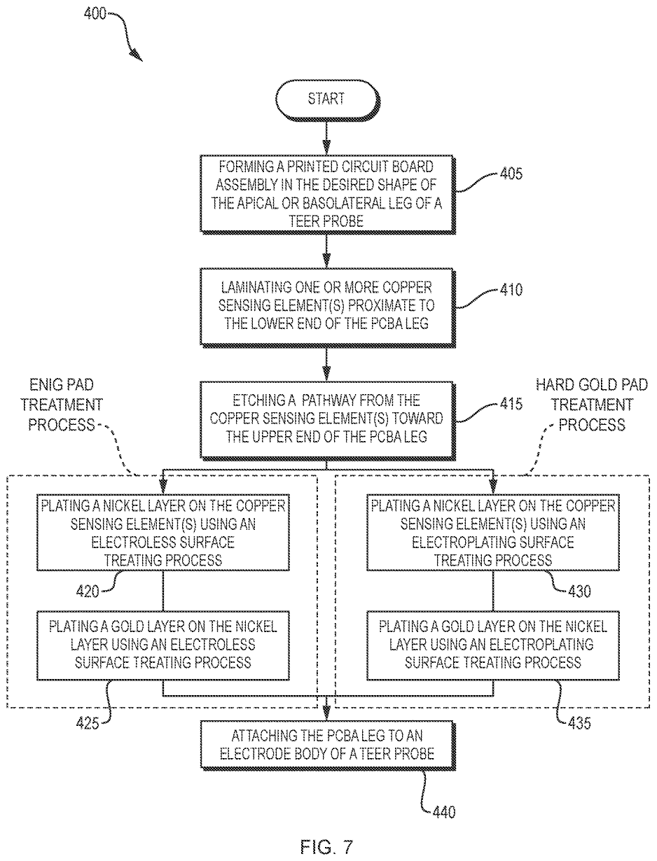

Turning to FIG. 7, illustrated is a flowchart 400 of the steps for formulating or manufacturing the PCBA apical legs 140, 240 and basolateral legs 150, 250 of the first and second embodiments of the probes 100, 200. At step 405, the PCBA's may be formed in the desired shape of the apical leg 140, 240 or basolateral leg 150, 250 of the TEER probes 100, 200. Next, at step 410, the formed PCBA's are then laminated with one or more copper sensing element(s) 144, 154, 246, 256 at locations that are proximate to the lower end 142, 152, 242, 252 of the PCBA legs 140, 150, 240, 250. At 415, one or more pathways may be etched from the copper sensing elements toward the upper ends 141, 151, 244, 254 of the PCBA legs 140, 150, 240, 250. As explained previously, and as depicted in FIG. 7, the PCBA legs 140, 150, 240, 250 may be treated via an ENIG pad treatment process or a hard gold pad treatment process. When treating the PCBA legs 140, 150, 240, 250 with the ENIG pad treatment process, at step 420, a layer of nickel is plated on the copper sensing elements 144, 154, 246, 256 using an electroless surface treating process. Furthermore, at step 425, a layer of gold is plated on the nickel layer using an electroless surface treating process. Conversely, when treating the PCBA legs 140, 150, 240, 250 with the hard gold pad treatment process, at step 430, a layer of nickel is plated on the copper sensing elements 144, 154, 246, 256 using an electroplating surface treating process. Furthermore, at step 435, a layer of gold is plated on the nickel layer using an electroplating surface treating process. Finally, at step 440, regardless of whether the PCBA legs 140, 150, 240, 250 were treated with the ENIG pad treatment process or the hard gold pad treatment process, the PCBA legs 140, 150, 240, 250 are then attached to the electrode body 120, 220 of the TEER probe 100, 200.

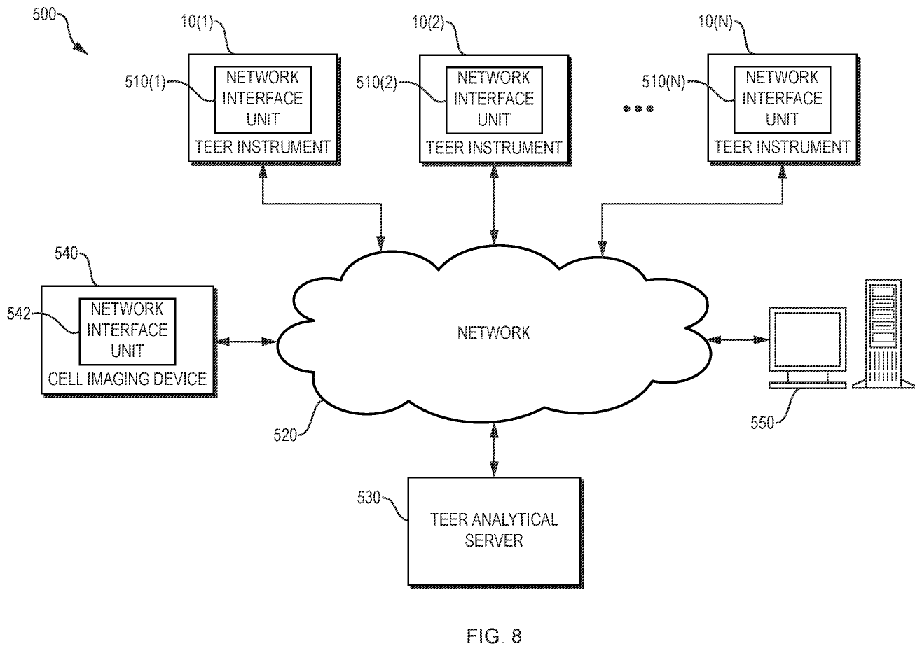

With reference to FIG. 8, depicted is a block diagram of an example network environment 500 in which the disclosed embodiments of the TEER instruments and other devices communicate with one another and/or a TEER analytical server 530. As depicted, TEER instruments 10(1)-10(N) (collectively “TEER instruments 10”) may each include network interface units 510(1)-510(N), respectively, and may be operated by local users in order to collect TEER and temperature measurements. The network interface (I/F) unit 510(1)-510(N) may, for example, be an Ethernet card or other interface device that allows the TEER instruments 10 to communicate over the communication network 520. Network I/F units 510(1)-510(N) may include wired and/or wireless connection capabilities, which enable each TEER instrument 10 (e.g., TEER instrument 10(1), TEER instrument 10(2), TEER instrument 10(N), etc.) to be capable and configured to send and/or receive data over a communication network 520, particularly with the TEER analytical server 530. The communication network 520 may include one or more wide area networks (WANs), such as the Internet, and one or more local area networks (LANs). Each TEER instrument 10 may be wired or wireless communication devices, such as, but not limited to the example embodiments depicted in FIG. 1, laptop and tablet computers, smartphones, etc.

FIG. 8 further discloses a cell imaging device 540 and a computing device 550. The cell imaging device 540 may be a device configured to utilize microscopy to monitor cells, measure cell confluency, measure cell count, and/or measure morphology. The cell imaging device 540 may also include a network interface unit 542 that enables the cell imaging device 540 to send and/or receive data over the communication network 520, and, particularly, with the TEER analytical server 530. The computing device 550 may be any wired or wireless communication devices capable of communicating over the communication network 520, such as, but not limited to desktop computers, laptop computers, tablet computers, smartphones, and the like.

In a transmit direction, TEER instruments 10 capture TEER measurements and/or temperature measurements via the probes 100, 200, 300(A), 300(B) described herein, encode the captured measurements into data packets, and transmit the data packets to the TEER analytical server 530, the computing device 550, and/or the cell imaging device 540. In some embodiments, the TEER instrument 10 may receive data packets from the TEER analytical server 530, other TEER instruments 10, the computing device 550, and/or the cell imaging device 540. Similarly, in a transmit direction, the cell imaging device 540 may capture microscopy images, encode the images into data packets, and transmit the data packets to the TEER analytical server 530, the computing device 550, and/or the TEER instruments 10. The cell imaging device 540 may also, in some embodiments, receive data packets from the TEER analytical server 530, the TEER instruments 10, the computing device 550, and/or other cell imaging devices.

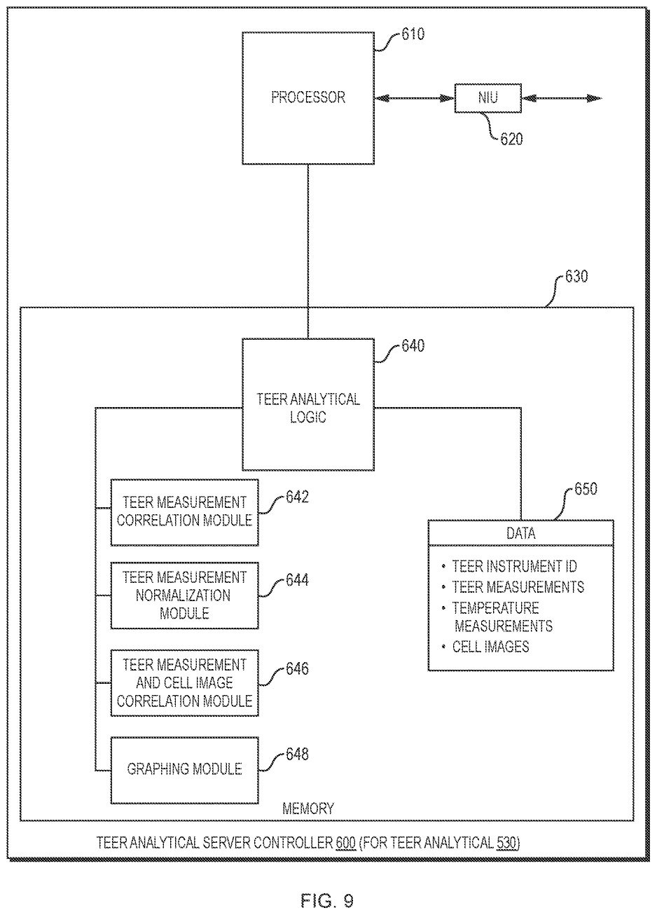

Reference is now made to FIG. 9, which depicts an example block diagram of a controller 600 of the TEER analytical server 530 configured to perform techniques for analyzing and correlating the measurements and images captured by the TEER instruments 10 and the cell imaging device 540. There are numerous possible configurations for controller 600 and FIG. 9 is meant to be an example. The controller 600 includes a processor 610, a network interface unit 620, and memory 630. The network interface (I/F) unit (NIU) 620 is, for example, an Ethernet card or other interface device that allows the controller 600 to communicate over communication network 520. Network I/F unit 620 may include wired and/or wireless connection capability.

Processor 610 may include a collection of microcontrollers and/or microprocessors, for example, each configured to execute respective software instructions stored in the memory 630. Portions of memory 630 (and the instruction therein) may be integrated with processor 610. In the transmit direction, processor 610 encodes TEER measurements and temperature measurements captured by TEER instruments 10, as well as microscopy data from the cell imaging device 540, encodes the captured measurements and images into data packets, and causes the encoded data packets to be transmitted to communication network 520. In a receive direction, processor 610 decodes measurement data packets and imaging data packets received from communication network 520 and sends the decoded data packets to the software instructions.

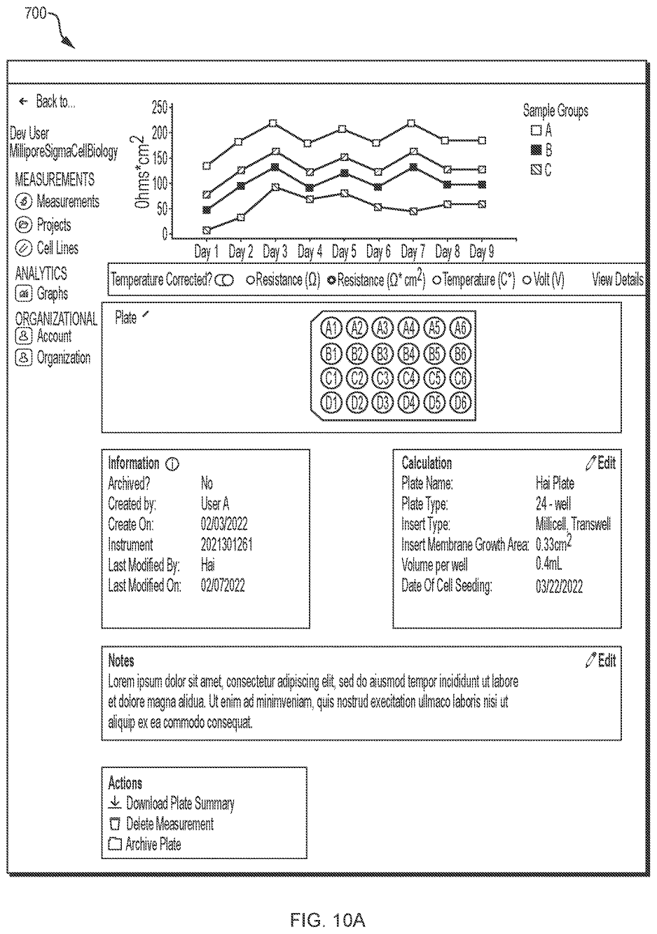

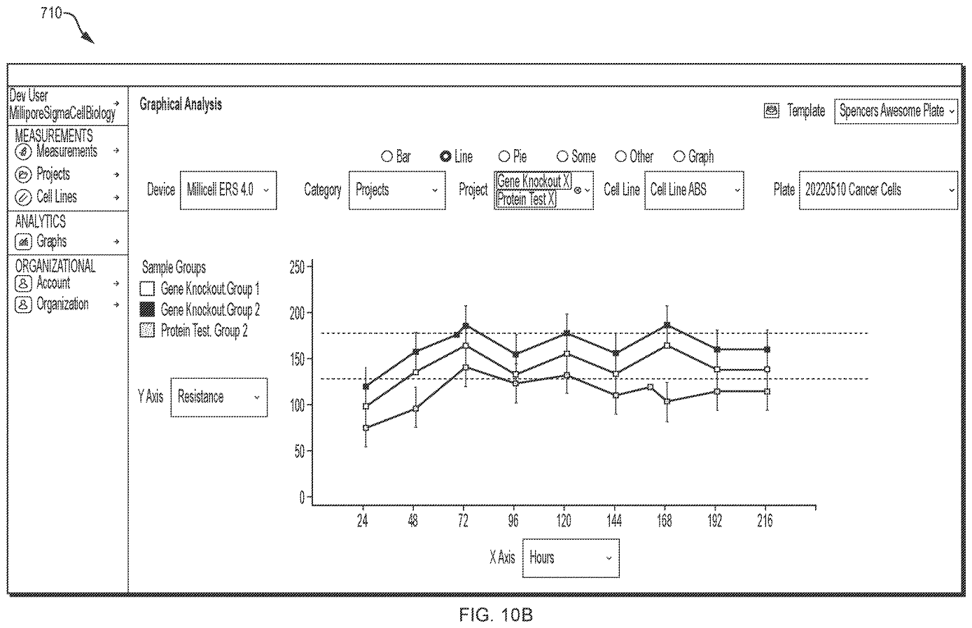

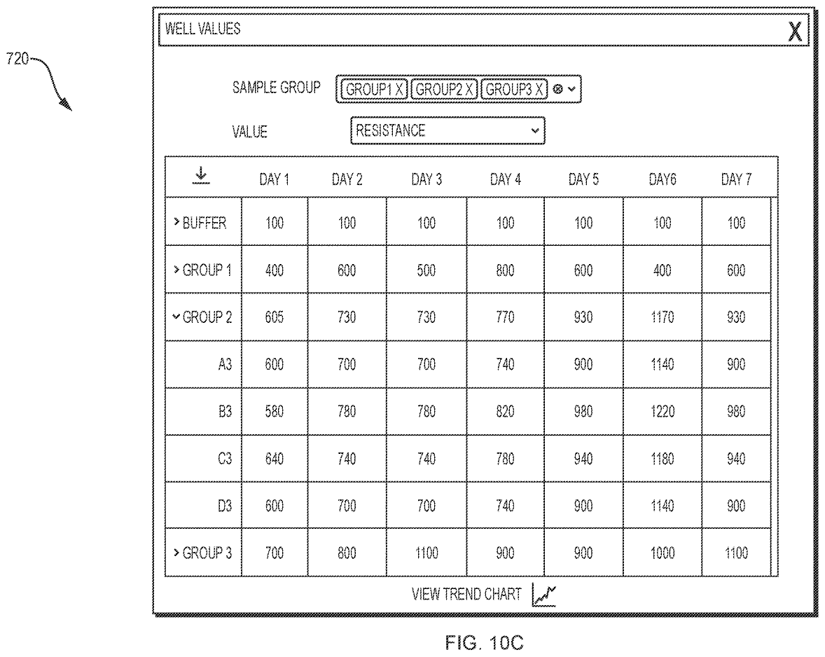

The memory 630 may include read only memory (ROM), random access memory (RAM), magnetic disk storage media devices, optical storage media devices, flash memory devices, electrical, optical, or other physical/tangible (e.g., non-transitory) memory storage devices. Thus, in general, the memory 630 may comprise one or more computer readable storage media (e.g., a memory device) encoded with software comprising computer executable instructions and when the software is executed (by the processor 610) it is operable to perform the operations described herein. For example, the memory 630 stores or is encoded with instructions for TEER analytical logic 640 that facilitates the correlation of the TEER measurements with the temperature measurements, normalizes the TEER measurements, correlates the TEER measurements with the cell microscopy images, and generates graphical outputs that may utilized the normalized and correlated data. TEER analytical logic 640 includes the TEER measurement correlation module 642 configured to correlate the TEER measurements collected via the probes 100, 200, 300(A), 300(B) with the temperature measurements collected via the probes 100, 200, 300(A), 300(B), the TEER measurement normalization module 644 configured to normalize the TEER measurements based on the correlated temperature measurements, the TEER measurement and cell image correlation module 646 configured to correlate the TEER measurements collected via the probes 100, 200, 300(A), 300(B) of the TEER instruments 10 with the microscopy images collected via the cell imaging device 540, and a graphing module 648 configured to utilize the TEER measurements, the temperature measurements, and/or the microscopy images to generate graphical images like those depicted in FIGS. 10A-10C for analysis.

In addition, memory 630 may store data 650 used and generated by logic/modules 640-648, including, but not limited to: TEER instrument identification (i.e., which TEER device recorded the measurement), TEER measurements, temperature measurements, and/or cell images.

With reference to FIGS. 10A-10C, and as previously explained, illustrated are example embodiments of graphical images that may be generated by the TEER analytical logic 640 of the TEER analytical server 530. FIG. 10A illustrates a first example embodiment of a graphical user interface 700 (hereinafter “GUI”) generated by the TEER analytical logic 640 of the TEER analytical server 530. The GUI 700 depicts a line graph of a resistance area (ohms*cm²—along the y-axis) vs. the day for three sample groups (i.e., A, B, C). The GUI 700 allows for a user to view the data as being temperature corrected, and provides an ability for the user to change the y-axis to represent resistance (ohms), resistance area (ohms*cm²), temperature (degrees Celsius), and voltage (V). The GUI 700 further depicts a graphical image of the culture plate (e.g., 24-well culture plate) from which the measurements were collected, where each well of the culture plate are labeled. The GUI 700 provide additional information such as, but not limited to, whether or not the data has been archived, the user that created the data, the date on which the data was created, the instrument ID that performed the measurements for the data, the user that last modified the data, and the date on which that user modified the data. The GUI 700 also enables a user to provide additional data pertaining to the calculations, such as, but not limited to, the name of the culture plate from which the measurements were taken, the type of culture plate (e.g., a 24-well culture plate), the type of insert placed within the wells of the culture plate, the growth area of the insert membrane, the volume per well, and the date of cell seeding. Finally, the GUI 700 allows for the users to input their own notes into that pertain to the data, as well as the ability to download a summary of the data, delete the measurement data, and/or archive the data.

Turning to FIG. 10B, illustrated is another example GUI 710 that provides a user with the ability to perform a graphical analysis of the data. While GUI 710 depicts a graph of the measurements of three sample groups as a line graph, the GUI 710 provides the user with the ability to depict the data as a bar graph, a line graph (as selected), a pie chart, along with other graphical options. The GUI 710 also enables a user to select which data is represented on the y-axis and the x-axis. In the example depicted, the user has selected to view resistance (y-axis) over hours (x-axis). Finally, the GUI 710 allows a user to analyze and filter the data by device, category, projects, cell lines, and plates.

Turning to FIG. 10C, illustrated is yet another example GUI 720 that presents a graphical analysis of the data. The GUI 720 depicted in FIG. 10C contains a chart that depicts various values (in this instance, resistance) for a series of wells of a culture plate (or just the culture plate itself) over a period of several days (i.e., 7 days). The user was able to choose three groups to be displayed in comparison with one another and with the buffer values over the same time period. The GUI 720 also enables a user to expand and collapse the various wells of a particular group, add or remove sample groups, and change the value that is displayed.

By moving away from silver/silver chloride electrodes to more robust and galvanically inert surface materials, the need for periodic re-treatment of the probes 100, 200, 300(A), 300(B) with sodium hypochlorite or hydrochloric acid is eliminated. Moreover, the integration of the temperature sensors 160, 260, 360 on the apical legs 140, 240, 340 of the probes 100, 200, 300(A), 300(B), respectively, enables users of the TEER system 10 to correlate TEER measurements to temperatures. This further allows users and/or systems to normalize TEER measurements based on its correlating temperature measurement in order to improve accuracy of the TEER measurements and experimental results. In addition, by integrating the TEER system with the ability to communicate with a cloud computing environment over a network, the TEER system allows for: easy data transfer from the TEER instrument to a server and/or personal computer for documentation and analysis; advanced data analytics and graphing optimized for TEER measurements; the ability to apply a temperature compensation to TEER data based on temperature sensor readings; and the ability to correlate TEER data with microscopy images of cells.

While the apparatuses presented herein have been illustrated and described in detail and with reference to specific embodiments thereof, it is nevertheless not intended to be limited to the details shown, since it will be apparent that various modifications and structural changes may be made therein without departing from the scope of the inventions and within the scope and range of equivalents of the claims. For example, the probes presented herein may be modified to contain any number of housings, components/bodies, legs sensing elements, temperature sensors, etc.

In addition, various features from one of the embodiments may be incorporated into another of the embodiments. That is, it is believed that the disclosure set forth above encompasses multiple distinct inventions with independent utility. While each of these inventions has been disclosed in a preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Accordingly, it is appropriate that the appended claims be construed broadly and in a manner consistent with the scope of the disclosure as set forth in the following claims.

It is also to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment of the invention. Additionally, it is also to be understood that the components of the bioprocessing system described herein, the manifold assembly described herein, or portions thereof may be fabricated from any suitable material or combination of materials, such as, but not limited to, thermoplastics, plastics, or metals (e.g., copper, bronze, aluminum, steel, etc.), as well as derivatives thereof, and combinations thereof. In addition, it is further to be understood that the steps of the methods described herein may be performed in any order or in any suitable manner.

Finally, when used herein, the term “comprises” and its derivations (such as “comprising,” etc.) should not be understood in an excluding sense, that is, these terms should not be interpreted as excluding the possibility that what is described and defined may include further elements, steps, etc. Similarly, where any description recites “a” or “a first” element or the equivalent thereof, such disclosure should be understood to include incorporation of one or more such elements, neither requiring nor excluding two or more such elements. Meanwhile, when used herein, the term “approximately” and terms of its family (such as “approximate,” etc.) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies, etc. The same applies to the terms “about,” “around,” “generally,” and “substantially.”

What is claimed is:

1. A probe for a trans-epithelial electrical resistance and trans-epithelial potential difference instrument, the probe comprising:
an upper housing;
an electrode body coupled to the upper housing:
an apical leg coupled to the electrode body;
a basolateral leg coupled to the electrode body; and
a temperature sensor disposed proximate to the apical leg or the basolateral leg.

2. The probe according to claim 1, wherein the temperature sensor is surface mounted on the apical leg and coated with an electrically isolating coating.

3. The probe according to claim 1, wherein the temperature sensor is an encapsulated thermistor or a thermocouple.

4. The probe according to claim 1, wherein the apical leg has an upper end, an opposite lower end, and at least one sensing element disposed proximate to the lower end.

5. The probe according to claim 4, wherein the temperature sensor is disposed proximate to the at least one sensing element.

6. The probe according to claim 4, further comprising:
a tube disposed in proximity to the apical leg, the tube having an upper end and a lower end, where the lower end of the tube is disposed in proximity to the lower end of the apical leg.

7. The probe according to claim 6, wherein the temperature sensor is disposed within the lower end of the tube.

8. The probe according to claim 7, wherein the tube is coupled to the electrode body.

9. The probe according to claim 7, wherein the tube is coupled to the apical leg.

10. The probe according to claim 9, wherein the apical leg further comprises a channel and the tube is disposed within the channel of the apical leg.

11. The probe according to claim 1, wherein the instrument comprises a network interface unit configured to send measurements taken via the probe to an analytical server over a network.

12. A probe for a trans-epithelial electrical resistance and trans-epithelial potential difference instrument, the probe comprising:
an upper housing;
an electrode body coupled to the upper housing;
an apical leg coupled to the electrode body; and
a basolateral leg coupled to the electrode body, wherein the apical leg and the basolateral leg are printed circuit board assemblies.

13. The probe according to claim 12, wherein the apical leg has an upper end, an opposite lower end, at least one copper sensing element disposed proximate to the lower end, wherein the basolateral leg has an upper end, an opposite lower end, at least one copper sensing element disposed proximate to the lower end, and wherein the at least one copper sensing elements of the apical leg and the basolateral leg are coated with a layer of nickel and a layer of gold via an electroless nickel, immersion gold treatment process.

14. The probe according to claim 13, wherein the instrument comprises a network interface unit configured to send measurements taken via the probe to an analytical server over a network.

15. The probe according to claim 12, further comprising:
a temperature sensor disposed proximate to the apical leg or the basolateral leg.

16. A probe for a trans-epithelial electrical resistance and trans-epithelial potential difference instrument, the probe comprising:
an upper housing;
an electrode body coupled to the upper housing;
a tubular apical leg coupled to the electrode body; and
a tubular basolateral leg coupled to the electrode body, wherein the tubular apical leg and the tubular basolateral leg constructed from stainless steel.

17. The probe according to claim 16, wherein the tubular apical leg and the tubular basolateral leg are constructed from citric passivated 304 stainless steel.

18. The probe according to claim 16, wherein the instrument comprises a network interface unit configured to send measurements taken via the probe to an analytical server over a network.

19. The probe according to claim 16, wherein the tubular apical leg has an upper end, an opposite lower end, and a conduit spanning from the upper end to the lower end.

20. The probe according to claim 19, further comprising:
a temperature sensor disposed within the conduit of the tubular apical leg proximate to the lower end.
